(19) United States
(12) Patent Application Publication
(10) Pub. No.: US 2011/0184651 A1
(43) Pub. Date: Jul. 28, 2011

(54) ELECTRONIC ASSAY APPARATUS AND METHOD THEREOF

(76) Inventors: Shih-Yi WENG, Jinning Township (TW); Tien-Rung Tsai, Taipei (TW); Woei-Yuh Lee, Taipei City (TW); Chao-Wang Chen, Taipei City (TW)

(21) Appl. No.: 12/693,471

(22) Filed: Jan. 26, 2010

(51) Int. Cl. G01N 33/50; G06F 19/00

(52) U.S. Cl. 702/19; 702/22

(57) ABSTRACT

The present invention related to an electronic assay apparatus and a testing method thereof for increasing efficiency and saving power. The electronic assay apparatus for determining a result of an assay performed using a test strip comprises two light sources, one detector and a microprocessor. The two light sources respectively illuminate light incident upon a test zone or a control zone of a test strip. The only one detector disposed between the two light sources and detects light reflected from the test zone and the control zone alternately. The microprocessor compares a calculating result value to only one threshold for showing a result.

### BACKGROUND OF THE INVENTION

1. Field of the Invention

The present invention relates in general to an electronic assay apparatus and a method thereof, which are adapted for quickly and simply reading assay result.

2. Description of the Related Art

Lateral flow test strips are known in the art and may be used in clinical diagnosis to determine the presence of an analyte of interest in a sample, such as a bodily fluid. For example, a lateral flow test strip suitable for the measurement of the pregnancy hormone human chorinic gonadotropin (hCG) is widely commercially available. The test strip usually has a control zone for determining the work of the assay and has a test zone for determining the presence of the interested analyte. However, such commercially available strips require the result to be interpreted by the user. Sometimes the result will be erroneous due to a degree of subjectivity, for example, different users may obtain different test results especially when a single color test zone is light and blur, which is undesirable.

Electronic assay apparatuses for reading the result of the test strips are known. A conventional electronic assay apparatus includes a test strip and a circuit board. The test strip is positioned related to the circuit board and has a control zone and a test zone. The circuit board includes a microprocessor, three light sources disposed thereon and two light detectors correspondingly disposed beneath the first light source and the third light source, respectively. The control zone and the test zone are aligned to the first and the third light sources respectively so as to allow light from the sources to illuminate on respectively. The second light source illuminates a reference zone between the control zone and the test zone to obtain a background. The first light detector detects light reflected from the control zone and some of the reference zone and the second light detector tests light reflected from some of the reference zone and the test zone. The microprocessor receives a detection signal from the light detectors for determining response circumstances of the control zone and the test zone. Furthermore, the microprocessor also detects the rate of change of reading with respect to time, or d (reading)/d (time) to calculate the result. Alternatively, the rate of change of slope with respect to time may be measured or calculated by d²(reading/d)time)². The result is positive when the reading value exceeds an upper threshold and is negative when the reading value lowers a lower threshold.

However, such the conventional electronic assay apparatus disadvantageously requires three light sources and two light detectors, which require much cost and more complicated structure. In addition, the usual used light source is red which needs more power to work and requires much cost. Further, the conventional electronic assay apparatus needs to detect the flow rate and it is more complicated to read the result. Thus, a need exists for improved electronic assay apparatus.

### SUMMARY OF THE INVENTION

According to one aspect of the present invention is to provide an electronic assay apparatus that comprises simple light sources and detector to achieve result reading. In a preferred embodiment of the present invention, the electronic assay apparatus for determining the result of an assay performed using a test strip, the apparatus comprises:

a circuit board comprising:

a first light source illuminating light incident upon a test zone of the test strip;

a second light source illuminating light incident upon a control zone spatially separated from the test zone of the test strip;

only one detector disposed between the first light source and the second light source to detect light reflected from the test zone and the control zone alternately and generating signals responsive to the test zone and the control zone; and

a microprocessor for receiving the signals from the detector and calculating the signals to a result value;

wherein the microprocessor compares the result value to a threshold and generates an output signal if the result value exceeds the threshold and indicative of a first result, or, alternatively, the output signal indicative of a second result if the result value is less than the threshold.

The apparatus in accordance with the present preferred further comprises a baffle connected with the circuit board and comprises a plurality of shelters defining a plurality of openings corresponding to the light sources and the detector. More preferably, the plurality of shelters comprise a first shelter, a second shelter, a third shelter and a fourth shelter. In a preferred embodiment, the first shelter and the second shelter defined respectively corresponding to outside of the first light source and the second light source to block outside light source, and the third shelter and the fourth shelter respectively formed between the first shelter and the second shelter and defined three openings corresponding to the first light source, the detector and the second light source.

The baffle employs in the present invention further can comprise a blocker so sized and positioned as to prevent direct light from the first light source and the second light source. Preferably, the blocker defined two slits respectively formed between the third shelter and the blocker and between the fourth shelter and the blocker for permitting the detector to detect the reflected light from the test zone and the control zone respectively.

The apparatus in accordance with the present invention preferably further comprises an ejective element connected with the baffle for ejecting the test strip.

Furthermore, the circuit board can further comprises a switch cooperated with the ejective element for activating the microprocessor when the test strip inserted.

In a preferred embodiment of the present invention, the microprocessor calculated a difference value between the signals of the control zone and the test zone. Preferably, the difference value is calculated by

R=(Tₘₐₓ−Tₘᵢₙ)/(Cₘₐₓ−Cₘᵢₙ);

D_{f}=C_{b}−(T_{b}*R); and

V=T_{final}*R+D_{f}−C_{b}

Where R is a difference ratio,

T and C are respectively test zone and control zone measurements,

max is the detected maximum value,

min is the detected minimum value,

b is the detected background value,

D_{f} is a drift value,

final is the final detected value, and

V is the result value.

Preferably, T_{b} and C_{b} are detected before a sample received in the test strip.

Another aspect of the present invention provides a method for testing an assay, which comprises:

positioning a test strip, having a test zone and a spatially separated control zone, in relation to an assay result reader, the reader comprising a cover enclosing a first light source, a second light source and only one detector;

receiving an assay sample;

measuring the light level received by the detector;

determining, using a microprocessor and based on the light level, a result of the assay performed on the test strip; and

displaying the result of the assay;

wherein:

the first light source is aligned for illuminating light incident upon the test zone of the test strip;

the second light source is aligned for illuminating light incident upon the control zone;

the detector is so positioned as to receive light reflected from the test zone and the control zone alternately; and

the microprocessor compares the result to a threshold and generates an output signal if the result value exceeds the threshold and indicative of a first result, or, alternatively, the output signal indicative of a second result if the result value is less than the threshold. Preferably, the first result is a negative result, and the second result is a positive result.

In a preferred embodiment of the present invention, the method can further comprise checking whether a calibration value existence before receiving the assay sample. Preferably, the calibration value is the lowest reading value detected by the detector. In another preferred embodiment of the present invention, the method can further comprise checking whether a signal detecting from the control zone higher than a predicted set value before receiving the assay sample. Preferably, the predicted value is a control calibration value added a fixed value.

The method in accordance with the present invention preferably further comprises detecting a background of the test zone T_{b} and the control zone C_{b} respectively before receiving the assay sample. Preferably, determining the result using:

R=(Tₘₐₓ−Tₘᵢₙ)/(Cₘₐₓ−Cₘᵢₙ);

D_{f}=C_{b}−(T_{b}*R); and

V=T_{final}*R+D_{f}−C_{b}

Where R is a difference ratio,

T and C are respectively test zone and control zone measurements,

max is the detected maximum value,

min is the detected minimum value,

b is the detected background value,

D_{f} is a drift value,

final is the final detected value, and

V is the result value.

The method in accordance with the present invention can further comprise:

detecting reflected light from the control zone in a predetermined period of time and showing an error if the detected reflected light is quiet the same.

Preferably, the method further comprises ejecting the test strip after displaying the result and terminating the assay.

The electronic assay apparatus and method in accordance with the present invention has following advantages.

1. The electronic assay apparatus in accordance with the present invention employs green light, blue light or yellow green light to save the cost and have high efficiency.

2. The electronic assay apparatus in accordance with the present invention employs green light, blue light or yellow green light to detect red color for decreasing noise compared with red light to detect blue color.

3. The electronic assay apparatus in accordance with the present invention does not need to set a further light source for illuminating a reference zone of the test strip so it can decrease processing step and decrease structure complexity.

4. The electronic assay apparatus in accordance with the present invention calculates a result value and compares the result value to only one threshold so the operational process is simple and the result is clear and definite.

5. The pregnancy testing kit employed in the present invention comprises one electronic assay apparatus and a plurality of test strip sets for conveniently users reusing the electronic assay apparatus comparing to conventional unusable electronic assay apparatus.

Further scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1 is a perspective view of an electronic assay apparatus in accordance with the present invention;

FIG. 2 is a perspective exploded view of the electronic assay apparatus of FIG. 1 and a test strip set in accordance with the present invention;

FIG. 3 is a partial cross-sectional view of certain internal components showing an embodiment of one arrangement of the electronic assay apparatus of FIG. 1;

FIG. 4 is a preferred embodiment of a block diagram of the electronic assay apparatus of FIG. 1; and

FIG. 5 is a preferred flow chart illustrating a method of reading an assay result of the electronic assay apparatus of FIG. 1.

### DESCRIPTION OF THE PREFERRED EMBODIMENTS

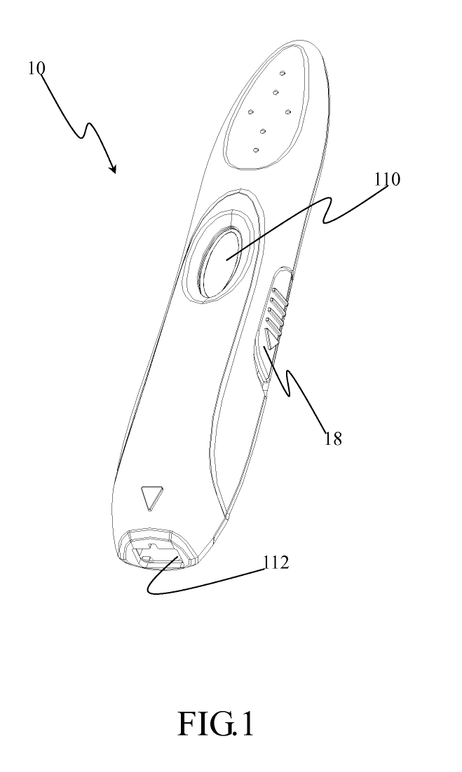

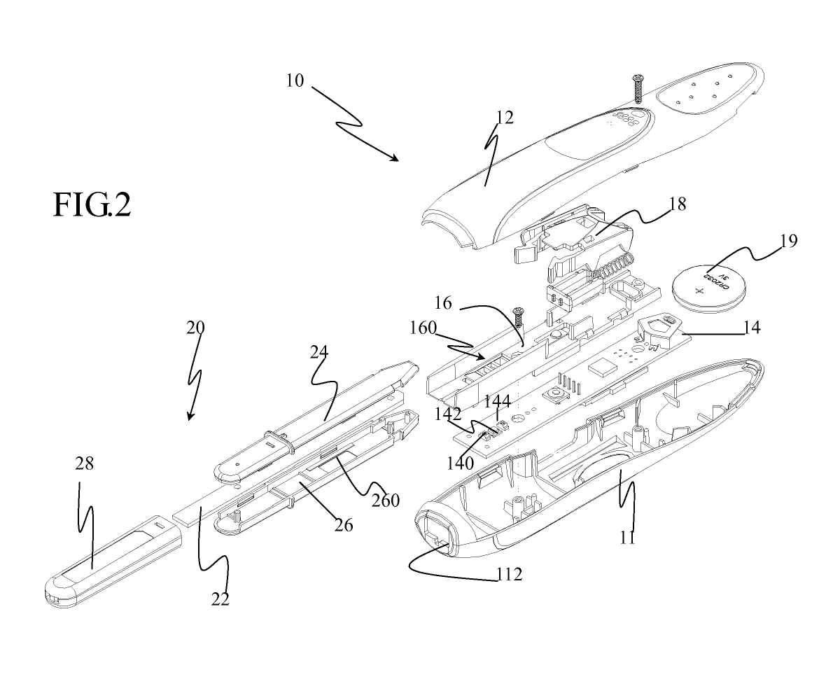

With reference to FIGS. 1 to 2, a preferred embodiment of an electronic assay apparatus in accordance with the present invention comprises a cover (10), a circuit board (14), a baffle (16) and an ejective element (18). The cover (10) comprises a top cover (11) and a bottom cover (12) covered with the top cover (11). The top cover (11) comprises a display (110) for displaying an assay result and an inserting opening (112) for inserting a test strip set (20).

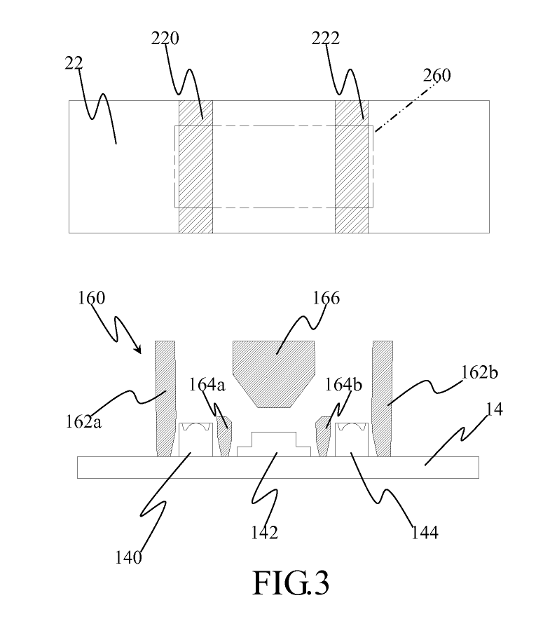

The test strip set (20) comprises a test strip (22) with a housing. The housing comprises a bottom housing (24), a top housing (26) and a lid (28). The bottom housing (24) and the top housing (26) connect together and the lid (28) covers one end of the connected bottom housing (24) and the top housing (26) to wrap the test strip (22). Please referred to FIG. 3, the test strip (22) comprises a test zone (220) and a control zone (222). The control zone (222) reflects whether a test is working and the test zone (220) tests a presence of an interested analyte. The top housing (26) comprises a window (260) for showing the test zone (220) and the control zone (222).

The circuit board (14) comprises a first light source (140), a detector (142) and a second light source (144). The first light source (140) and the second light source (144) disposed on the circuit board (14) for illuminating light to the test zone (220) and the control zone (222) respectively. Preferably, the first light source (140) and the second light source (144) are capable of emitting a green light, a blue light or a yellow green light. These light have more efficiency for working. The detector (142) disposed between the first light source (140) and the second light source (144) for detecting the reflected light from the test zone (220) and the control zone (222) and transferring signals responsive to the reflected light therefrom.

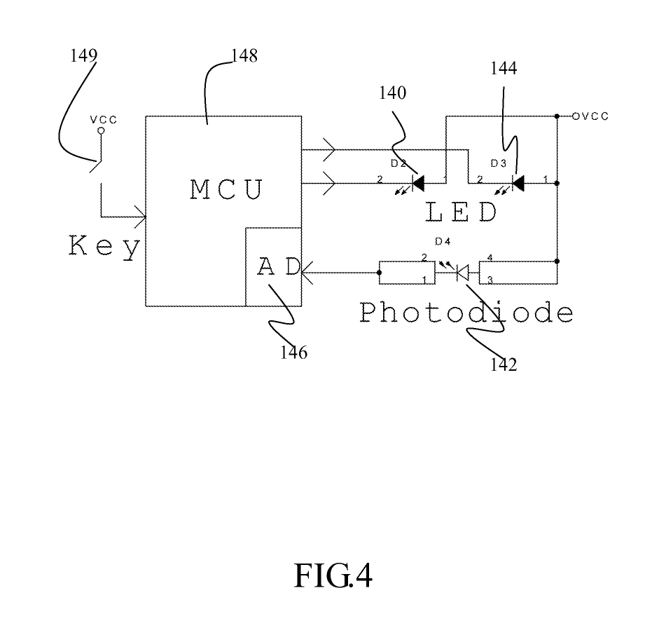

Further referring to FIG. 4, it is a preferred embodiment of a block diagram of the circuit board (14) in accordance with the present invention. The circuit board (14) comprises two light sources (140, 144). When a test strip is inserted into the apparatus, each light sources (140, 144) is aligned with a respective zone of the test strip. The first light source (140) is aligned with the test zone (220) and the second light source (144) is aligned with the control zone (222) (as shown in FIG. 3). The only one detector (142) detects light reflected from both zones and generates a current, the magnitude of which is proportional to the amount of light incident upon the detector (142). The current is converted into a voltage and fed into an analogue to digital converter (AD) (146). The resulting digital signal is read by a microprocessor (148). The microprocessor (148) switches on the light sources (140, 144) one at a time, so that only one of the two zones is illuminated at any given time. Furthermore, a switch (149) will be closed by insertion of the test strip into the apparatus and controlled by the ejective element (18) described later, and which activates the microprocessor (148).

The baffle (16) connected with the circuit board (14) and comprises a plurality of shelters (160) defining a plurality of openings corresponding to the light sources (140, 144) and detector (142). With further reference to FIG. 3, the plurality of shelters (160) comprise a first shelter (162a), a second shelter (162b), a third shelter (164a) and a fourth shelter (164b). The first shelter (162a) and the second shelter (162b) defined respectively corresponding to outside of the two light sources (140, 144) to block outside light source. The third shelter (164a) and the fourth shelter (164b) respectively formed between the first shelter (162a) and the second shelter (162b) and defined three openings corresponding to the first light source (140), the detector (142) and the second light source (144).

The baffle (16) further comprises a blocker (166) defined upside corresponding to the detector (142) for blocking direct light from the first light source (140) and the second light source (144) and defined two slits. The two slits respectively formed between the third shelter (164a) and the blocker (166) and between the fourth shelter (164b) and the blocker (166) for permitting the detector (142) to detect the reflected light from the test zone (220) and the control zone (222).

The ejective element (18) connected with the baffle (16) and within the cover (10). In the beginning, the test strip set locks with the ejective element (18) when the test strip set (20) inserts into the apparatus and then the ejective element (18) closes the switch (149) to activate the microprocessor (148). After the test is done, push the ejective element (18) to let the test strip set (20) leave out of the ejective element (18) and then the switch (149) is opened to inactivate the microprocessor (148) and the apparatus will shut down.

In a further preferred embodiment, the electronic assay apparatus in accordance with the present invention further comprises a power source (19) (as shown in FIG. 2, there is one button cells).

In a preferable embodiment, the test strip (22) is employed for pregnancy testing and comprises a sample pad, a conjugated pad, a reaction membrane and an absorbent pad. The conjugated pad comprises mobilized mAb (mouse Antibody) anti-Beta hCG conjugated with colloidal gold. The test zone (220) and the control zone (222) are disposed within the reaction membrane. The test zone (220) comprises a vertical line of an antibody to hCG, preferably an immobilized goat anti-Alpha hCG and the control zone (222) comprises immobilized goat anti Mouse IgG. When a sample is received from the sample pad, it will pass through the conjugated pad to bring the mobilized mAb anti-Beta hCG conjugated with colloidal gold, and then pass through the reaction membrane to react. If hCG exists in the sample, hCG combined with the mAb anti-Beta hCG conjugated with colloidal gold and combined with the immobilized goat anti Alpha hCG to form a sandwich Ag-Ab complex and display the red color. Whether the sample contains hCG or not, the mobilized mAb anti-Beta hCG will combine with the goat anti Mouse IgG at the control zone (222) to check whether the test is workable.

Since the test strip (22) uses red colloidal gold, the apparatus should employ a green, blue or yellow green light source, and therefore, it can decrease the noise signals compared with red light source illuminating blue color on the test strip as conventional used.

In use, a dry test strip set (20) (for example, prior to contacting the sample) is inserted into the apparatus, this closes the switch (149) to activate the apparatus, which then performs a series programs.

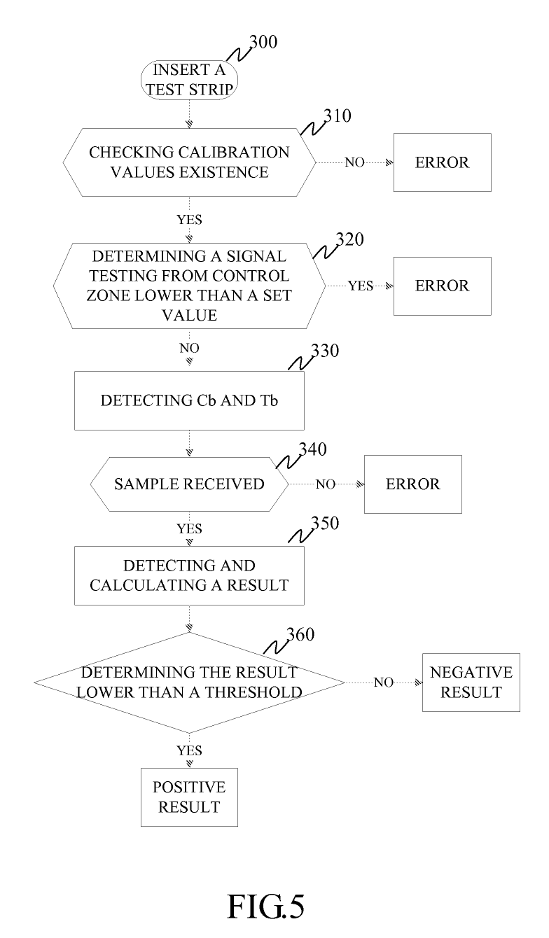

With further reference to FIG. 5, it is a preferred embodiment of a flow chart in accordance with the present invention showing a process of assay reading. When the test strip set inserts into the inserting opening of the apparatus, the apparatus is activated (step 300). Once the apparatus is activated, the microprocessor checks whether calibration values are existence or not (step 310) and if the response to this inquiry is positive, the “YES” branch is followed to step 320. A negative response to this inquiry results in the process that following the “NO” branch from step (310) to show an ERROR. In a preferred embodiment, the calibration values are set by manufacturer through testing the lowest reading value of the test zone and control zone respectively. The step 310 checks whether the apparatus is calibrated or not and it will exist the calibration values set in the apparatus if it is calibrated. Therefore, if there aren't the calibration values set in the apparatus that means the apparatus has not calibrated.

Step 320 is then determining whether a signal detecting from the control zone is lower than a predicted set value and if the response to this inquiry is negative, the “NO” branch is followed to step 330. A positive response to this inquiry results following the “YES” branch from step 320 to show an ERROR. For example, the predicted value is a control calibration value added three hundred and if the value detecting from the control zone is lower than the predicted value, it means the test strip was used or a battery is dead.

If step 310 and step 320 are all passed, step 330 detects C_{b} and T_{b} before sample received. C_{b} presents control zone background and T_{b} presents test zone background. In a preferred embodiment of the present invention, the background of C_{b} and T_{b} is calculated by the initial detected value before sample received. Therefore, the apparatus in accordance with the present invention does not need to comprise a further light source to illuminate a reference zone of the test strip and decreases detecting steps.

After sample receiving detected (step 340), step 350 is detecting signals by the detector from the control zone and test zone and calculating a result. For example, if the detected signals are suddenly decrease, it means the test strip is receiving sample and light is absorbing so the detected signals are decrease timely.

After a specific timed interval following sample received, desirably the measurements are taken at regular intervals. The measurements are made as a sequence of many readings over the specific periods of time and interleaved zone by zone. The microprocessor calculates the result by a series of equation.

Firstly, R (ratio) is calculated.

R=(Tₘₐₓ−Tₘᵢₙ/(Cₘₐₓ−Cₘᵢₙ); in which R is a difference ratio detected by the detector between the control zone and test zone. T and C presents respectively test zone and control zone measurements. Max means the detected maximum value and min means the detected minimum value.

Further, D_{f} will be calculated.

D_{f}=C_{b}−(T_{b}*R); in which D_{f} is a drift value.

Since the intensity of light output from different light sources is rarely identical, such variation could affect the apparatus reading result. Therefore, it is calculated the difference ratio and drift value detected between the control zone and the test zone to normalize the result so that the result respectively detected from the two zones will approximately based on an equal standard.

Then the result value (V) is calculated by the following equation:

V=T_{final}*R+D_{f}−C_{b}

After the predicted period of time, a final signal of the test zone is measured and the result value (V) is calculated according to the final signal.

Step 360 is determining whether the result value is lower than a threshold and if the response to this inquiry is positive, the “YES” branch is show a positive result. A negative response to this inquiry results in the process that following the “NO” branch to show a negative result.

Furthermore, if the detecting signals from the control zone in a predetermined period of time is quite the same that means the assay is invalid, the apparatus will show an error.

According to an embodiment of the present invention, the electronic assay apparatus is for pregnancy testing and the sample is urine. Preferably, the threshold is responsive to samples of urine containing hCG of a concentration of 25 mIU/ml. Since the apparatus according to the present invention detects the reflected light from the control zone and the test zone, the result value lower than the threshold means the sample contains hCG higher than 25 mIU/ml.

In a preferred embodiment of the present invention, a pregnancy testing kit is provided. The pregnancy testing kit comprises one electronic assay apparatus and a plurality of test strip sets for reusing the electronic assay apparatus.

Other embodiments of the invention will appear to those skilled in the art from consideration of the specification and practice of the invention disclosed herein. It is intended that the specification and examples to be considered as exemplary only, with a true scope and spirit of the invention being indicated by the following claims.

What is claimed is:

1. An electronic assay apparatus for determining a result of an assay performed using a test strip, the apparatus comprising:
a circuit board comprising:
a first light source illuminating light incident upon a test zone of the test strip;
a second light source illuminating light incident upon a control zone spatially separated from the test zone of the test strip;
only one detector disposed between the first light source and the second light source to detect light reflected from the test zone and the control zone alternately and generating signals responsive to the test zone and the control zone; and
a microprocessor for receiving the signals from the detector and calculating the signals to a result value;
wherein the microprocessor compares the result value to a threshold and generates an output signal if the result value exceeds the threshold and indicative of a first result, or, alternatively, the output signal indicative of a second result if the result value is less than the threshold.

2. The apparatus as claimed in claim 1, further comprising a baffle connected with the circuit board and comprising a plurality of shelters defining a plurality of openings corresponding to the light sources and the detector.

3. The apparatus as claimed in claim 2, wherein the plurality of shelters comprise a first shelter, a second shelter, a third shelter and a fourth shelter.

4. The apparatus as claimed in claim 3, wherein the first shelter and the second shelter defined respectively corresponding to outside of the first light source and the second light source to block outside light source.

5. The apparatus as claimed in claim 4, wherein the third shelter and the fourth shelter respectively formed between the first shelter and the second shelter and defined three openings corresponding to the first light source, the detector and the second light source.

6. The apparatus as claimed in claim 5, wherein the baffle further comprises a blocker so sized and positioned as to prevent direct light from the first light source and the second light source.

7. The apparatus as claimed in claim 6, wherein the blocker defined two slits respectively formed between the third shelter and the blocker and between the fourth shelter and the blocker for permitting the detector to detect the reflected light from the test zone and the control zone respectively.

8. The apparatus as claimed in claim 7, further comprising an ejective element connected with the baffle for ejecting the test strip.

9. The apparatus as claimed in claim 8, wherein the circuit board further comprises a switch cooperated with the ejective element for activating the microprocessor when the test strip inserted.

10. The apparatus as claimed in claim 1, wherein the first light source and the second light source illuminating green light, blue light or yellow green light for illuminating a red color on the test zone and the control zone respectively.

11. The apparatus as claimed in claim 1, further comprising a cover enclosing the circuit board.

12. The apparatus as claimed in claim 1, further comprising a display for displaying the result according to the microprocessor.

13. The apparatus as claimed in claim 1, wherein the microprocessor calculated a difference value between the signals of the control zone and the test zone.

14. The apparatus as claimed in claim 13, wherein the difference value is calculated by
R=(Tₘₐₓ−Tₘᵢₙ)/(Cₘₐₓ−Cₘᵢₙ);
D_{f}=C_{b}−(T_{b}*R); and
V=T_{final}*R+D_{f}−C_{b}
Where R is a difference ratio,
T and C are respectively test zone and control zone measurements,
max is the detected maximum value,
min is the detected minimum value,
b is the detected background value,
D_{f} is a drift value,
final is the final detected value, and
V is the result value.

15. The apparatus as claimed in claim 14, wherein T_{b} and C_{b} are detected before a sample received in the test strip.

16. The apparatus as claimed in claim 1, wherein the microprocessor switches on the light sources one at a time so that only one of the test zone and the control zone is illuminated at any given time and the detector detects a sequence of many readings over a specific periods of time and interleaved zone by zone.

17. A method for testing an assay, comprising:
positioning a test strip, having a test zone and a spatially separated control zone, in relation to an assay result reader, the reader comprising a cover enclosing a first light source, a second light source and only one detector;
receiving an assay sample;
measuring the light level received by the detector;
determining, using a microprocessor and based on the light level, a result of the assay performed on the test strip; and
displaying the result of the assay;
wherein:
the first light source is aligned for illuminating light incident upon the test zone of the test strip;
the second light source is aligned for illuminating light incident upon the control zone;
the detector is so positioned as to receive light reflected from the test zone and the control zone alternately; and
the microprocessor compares the result to a threshold and generates an output signal if the result value exceeds the threshold and indicative of a first result, or, alternatively, the output signal indicative of a second result if the result value is less than the threshold.

18. The method as claimed in claim 17, further comprising:
checking whether a calibration value existence before receiving the assay sample.

19. The method as claimed in claim 18, wherein the calibration value is the lowest reading value detected by the detector.

20. The method as claimed in claim 17, further comprising:
checking whether a signal detecting from the control zone higher than a predicted set value before receiving the assay sample.

21. The method as claimed in claim 20, wherein the predicted value is a control calibration value added a fixed value.

22. The method as claimed in claim 17, further comprising:
detecting a background of the test zone T_{b} and the control zone C_{b} respectively before receiving the assay sample.

23. The method as claimed in claim 22, wherein determining the result using
R=(Tₘₐₓ−Tₘᵢₙ)/(Cₘₐₓ−Cₘᵢₙ);
D_{f}=C_{b}−(T_{b}*R); and
V=T_{final}*R+D_{f}−C_{b}
Where R is a difference ratio,
T and C are respectively test zone and control zone measurements,
max is the detected maximum value,
min is the detected minimum value,
b is the detected background value,
D_{f} is a drift value,
final is the final detected value, and
V is the result value.

24. The method as claimed in claim 23, further comprising:
detecting reflected light from the control zone in a predetermined period of time and showing an error if the detected reflected light is quiet the same.

25. The method as claimed in claim 23, further comprising ejecting the test strip after displaying the result and terminating the assay.

26. The method as claimed in claim 17, wherein the first result is a negative result, and the second result is a positive result.

27. A pregnancy testing kit comprising an electronic assay apparatus as claimed in claim 1 and a plurality of test strip with a test zone and a spatially separated control zone.
